(19) United States
(12) Patent Application Publication
(10) Pub. No.: US 2023/0343428 A1
(43) Pub. Date: Oct. 26, 2023

(54) METHOD AND APPARATUS FOR ADAPTIVE ACTIONS BASED ON TEMPORAL-BASED PREDICTIONS OF NON-COMPLIANCE

(71) Applicant: Optum , Inc., Minnetonka, MN (US)

(72) Inventors: Sriram N. Iyer, Excelsior, MN (US); Nicholas Knaff Walker, Eden Prairie, MN (US); R Arun Prasath, Hyderabad (IN); Ranraj Rana Singh, Mumbai (IN); Diana B. Quach, Las Vegas, NV (US)

(21) Appl. No.: 18/165,010

(22) Filed: Feb. 6, 2023

Related U.S. Application Data
(60) Provisional application No. 63/363,246, filed on Apr. 20, 2022.

(51) Int. Cl. G16H 20/00; G06N 5/022

(52) U.S. Cl. CPC G16H 20/00; G06N 5/022

(57) ABSTRACT

Methods, computer program products, and systems for generating adaptive temporal-based prediction scores representative of a likelihood of non-compliance with respect to a recommendation associated with a user identifier. One or more prediction-based actions can be executed based on the adaptive temporal-based prediction score satisfying a threshold.

### REFERENCE TO RELATED APPLICATIONS

This application claims priority to and the benefit of U.S. Provisional Application No. 63/363,246, filed Apr. 20, 2022, the entire contents of which are incorporated herein by reference.

### BACKGROUND

Technical challenges related to computational and other negative impacts of non-compliance with recommendations exist. Through applied effort, ingenuity, and innovation, the inventors have developed systems and methods that overcome such challenges. Some examples of these solutions are described in detail herein.

### BRIEF SUMMARY

In general, various embodiments of the present disclosure provide methods, apparatus, systems, computing devices, computing entities, and/or the like. In some embodiments, the disclosed methods, systems, and computer program products include receiving real-time recommendation data associated with a user profile identifier, wherein the real-time recommendation data (i) originates at least in part from a remote computing entity and (ii) comprises temporal sequence data that indicates (a) a recommendation for the user profile identifier, and (b) the recommendation associated with a content type; generating using a predictive machine learning model and based at least in part on (i) a duration of elapsed network time since provision of the recommendation and (ii) the content type associated with the recommendation, an adaptive temporal-based prediction score representative of a likelihood of non-compliance with respect to the recommendation, wherein the predictive machine learning model has been trained based at least in part on: (i) one or more features (a) associated with historical transaction data and (b) associated with the recommendation, or (ii) actions performed in accordance with the historical transaction data associated with the user profile identifier; and initiating the performance of one or more prediction-based actions based at least in part on the adaptive temporal-based prediction score.

### BRIEF DESCRIPTION OF THE DRAWINGS

Having thus described the disclosure in general terms, reference will now be made to the accompanying drawings, which are not necessarily drawn to scale, and wherein:

FIG. 1 provides an illustration of an example embodiment of the present disclosure.

FIG. 2 provides an illustrative schematic of an example computing entity according to one embodiment of the present disclosure.

FIG. 3 provides an illustrative schematic representative of an example computing entity that can be used in conjunction with embodiments of the present disclosure.

FIG. 4 illustrates an example prediction-based action generation process for use with embodiments herein.

FIG. 5 illustrates an example process for adjusting adaptive temporal-based prediction scores in accordance with embodiments herein.

FIG. 6 is an architectural diagram of an example compliance management system in accordance with embodiments herein.

FIG. 7 is a diagram showing an example training process in accordance with embodiments herein.

FIG. 8 is illustrates example features in accordance with embodiments herein.

FIG. 9 illustrates example impact data in accordance with embodiments herein.

FIG. 10 illustrates example adjustments to adaptive temporal-based prediction scores in accordance with embodiments herein.

FIG. 11 depicts a comparison of example adaptive temporal-based prediction scores for two different individuals in accordance with embodiments herein.

FIG. 12 depicts an example compliance interface in accordance with embodiments herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not indicate being based only on or based solely on the referenced element or elements unless so indicated. Like numbers refer to like elements throughout.

### I. OVERVIEW AND TECHNICAL IMPROVEMENTS

Embodiments herein provide for adaptive actions based on temporal-based predictions associated with non-compliance. In various contexts, non-compliance with a recommended action, by an individual, can result in wasted resources and other detrimental situations (e.g., physical or health-related harm to the individual or those surrounding an individual). When non-compliance is evident, yet an individual may still intend to comply with the recommended action, a compliance management computing entity may continuously waste resources through transmission of communications to a client computing entity associated with the individual. Such wasted resources include computing resources, network bandwidth, and processing resources dedicated to the preparation and storage of data used in conjunction with the transmissions. Moreover, when non-compliance is an end result, unnecessary transformation and storage of data by the compliance management computing entity may occur because related systems track compliance or may be tangentially related to compliance. Additionally, non-compliance with certain recommendations can lead to wasted resources when non-compliance leads to physical harm to an individual (e.g., non-compliance with medical recommendations leading to hospitalization or further more severe care required). Finally, non-compliance at a single point in time for a particular individual may not be indicative of an actual likelihood of non-compliance (e.g., the likelihood of non-compliance may change over time and with provision of additional recommendations).

Embodiments of the present disclosure overcome the aforementioned drawbacks and more by enabling an adaptive temporal-based prediction score that indicates a likelihood of non-compliance with a recommendation with respect to real-time recommendation data. The predictive machine learning model may be trained based on one or more features (e.g., including temporal features) associated with historical transaction data and the recommendation and/or compliance by the individual in accordance with past recommendations. The adaptive temporal-based prediction score is updated, by the predictive machine learning model, based on temporal feature set progression during an entirety of a duration of elapsed network time. Accordingly, at any given point in time, and for a particular individual, a likelihood of non-compliance can be quantified such that mitigating actions can be automatically executed to improve the likelihood of compliance while also avoiding wasted resources, computational load on the overall system, and possibility of harm. Moreover, the real-time adaptation of actions based on real-time generations of adaptive temporal-based prediction scores enables the mitigating actions to be triggered nearly instantaneously as compared to a manual approach—where a manual approach may lead to discovery of a problem beyond the time when mitigating actions remain available (e.g., when the data is no longer meaningful or relevant).

The terms “recommendation” or “recommendation data structure” may refer to one or more items of data including a plurality of data records, where the plurality of data records describe or store data related to a suggested, proposed, and/or prescribed course of action to be completed associated with a user profile identifier (e.g., associated with an individual). Each data record of the plurality of data records may store features associated with the overall recommended action, as well as a user profile identifier (e.g., one or more items of data by which a user profile or individual may be uniquely identified). The recommendation may be generated, issued, and/or transmitted by a recommending computing entity and may be associated with a recommendation timestamp (e.g., a point of network time at which the recommendation data structure was generated). A recommendation may include or indicate one or more recommended actions (e.g., to be performed by the individual), including one or more initial actions and/or one or more follow-up or following actions. The individual may follow a recommendation by performing an action in accordance with the recommendation (e.g., by performing the suggested course of action of the recommendation). Alternatively, the individual may demonstrate inaction, non-compliance, or partial compliance with respect to the recommendation (e.g., by not performing, or by not fully performing the course of action of the recommendation). Various embodiments are applicable to different types of recommendations in different contexts, including healthcare, e-commerce, various professional disciplines involving expert recommendations, education, mental health counseling, consulting, and/or public health administration, to list a few examples. In some examples in the healthcare domain, recommendations may include filling or picking up a prescription or prescription refill, scheduling or attending an appointment (e.g., mammograms, colonoscopies, physical therapy, specialty referrals), scheduling or attending a follow-up appointment, and/or the like.

The term “recommending computing entity” may refer to a computing system configured to generate recommendations. The recommendations may be generated by the recommending computing entity based on certain data available to the recommending computing entity (e.g., data concerning the individual, data pertinent to a situation of the individual, received user input from a technician operating the recommending computing entity).

The term “duration of elapsed network time” may refer to a difference between a first network timestamp (e.g., a recommendation timestamp) and a second, subsequent, network timestamp (e.g., a timestamp associated with generation of an adaptive temporal-based prediction score).

The terms “recommendation compliance” or “compliance” may refer performance or completion of one or more actions in accordance with a recommendation. With respect to an individual recommendation, recommendation compliance may refer to the individual for whom the individual recommendation was issued performs (or is likely to perform) an action in accordance with the individual recommendation. According to various embodiments of the present disclosure, a high degree of compliance may be desirable (e.g., compliance), while a low degree of compliance (e.g., partial or incomplete compliance or non-compliance) may be undesirable.

The terms “prediction-based actions” or “mitigating actions” may refer to actions aimed at improving recommendation compliance in a given context. Operations aimed at improving recommendation adherence or compliance for an individual and/or a group of individuals may include generating system alerts and reminders, generating and/or sending electronic communications and/or reminders, displaying and/or gathering data relevant to improving adherence or compliance (e.g., more detailed data about the recommendations, data concerning the individual's intentions and/or motivations with respect to the recommendations), automated scheduling and calendaring, and/or generating alternative recommendations for the individuals. In one example, the prediction-based actions may include rendering of a compliance interface (and/or other screens/panes) via a display of a client computing entity.

For the purposes of illustration, in one example, the recommendation may be a prescription (e.g., a formal direction or order determined by a healthcare provider such as a doctor concerning medication to be taken by the individual). In this case, the recommending computing entity may be a computing system operated by the healthcare provider, the individual may be a patient undergoing treatment by the healthcare provider, and a time of issuance may be a point in time when the recommending computing entity generates the prescription (e.g., based on user input received from the healthcare provider) and/or communicates the prescription to the patient and/or related entity such as a pharmacy. Here, the course of action of the recommendation may be represented by a medication identified in the prescription, and performing an action in accordance with the recommendation may include picking up the medication at a pharmacy and/or administering the medication as instructed in the prescription. On the other hand, in this example, demonstrating non-compliance with respect to the recommendation may include the patient failing to pick up and/or administer the medication and/or communicating to a pharmacy or the healthcare provider that they do not intend to pick up and/or administer the medication.

In a similar example, the recommendation may be a referral (e.g., a formal direction determined by a healthcare provider such as a doctor concerning an additional provider to be consulted by the individual and/or an additional service, test, procedure, and/or consultation to be completed by the individual). A referral may be for a specialist consultation, a course of therapy services (e.g., physical therapy, mental health therapy), diagnostic testing (e.g., blood work, imaging), and/or, diagnostic procedures (e.g., colonoscopy, mammography), or the like. In this case, performing an action in accordance with the recommendation may include scheduling and/or attending an appointment with a different healthcare provider than that of the recommending computing entity (e.g., a specialist identified in the referral/recommendation), scheduling and/or attending one or more sessions or appointments with respect to services indicated in the referral (e.g., physical therapy), and/or scheduling and/or attending one or more appointments associated with tests or procedures recommended in the referral. In one embodiment, tracking recommendation compliance may include determining whether an individual is likely to demonstrate inaction with respect to the recommendation and/or one or more actions indicated in the recommendation, which may include the patient failing to schedule such appointments and/or sessions for said consultations, services, tests, and/or procedures and/or the patient failing to show up to such appointments and/or sessions when they are scheduled.

In another example, the recommendation may be a follow-up recommendation with respect to a previously issued recommendation, such as a prescription refill. Here, a time of issuance of the recommendation may be a point in time when the follow-up recommendation is determined to be due (e.g., a calculated refill due date based on dosage, administration, and/or refill data included in an originally-issued prescription) and/or when availability of the refill has been communicated to the patient. The course of action of the recommendation and any follow-up recommendations may be represented by a medication identified in an original prescription. Performing an action in accordance with the follow-up recommendation may include picking up not just the initial course of medication provided in the original prescription, but any refills of the medication and/or administering the refilled medication as instructed in the prescription for the duration of one or more refill periods indicated in the original prescription. On the other hand, in this example, demonstrating non-compliance with respect to the recommendation may include the patient failing to pick up and/or administer the refilled medication and/or communicating to a pharmacy or the healthcare provider that they do not intend to pick up and/or administer the refilled medication (e.g., even after having initially picked up and/or administered the medication as prescribed in the original prescription).

The term “adaptive temporal-based prediction score” may refer to a score associated with a recommendation and a user profile identifier, where the adaptive temporal-based prediction score is generated using a predictive machine learning model. The adaptive temporal-based prediction score may include a value that represents a likelihood that a recommendation will not be performed (e.g., percentage, probability value ranging from 0 to 1) with respect to one or more actions indicated in the recommendation. In one example, a minimal value (e.g., 0%, 0) represents that non-compliance with respect to the recommendation is an impossibility, a maximal value (e.g., 100%, 1) represents that non-compliance with respect to the recommendation is a certainty, and any intermediate value (e.g., between the minimal value and the maximal value) proportionally represents the likelihood of non-compliance with respect to the recommendation (e.g., such that the higher the value the more likely it is that the recommendation will not be performed).

The adaptive temporal-based prediction score may be an adaptive score that changes over time and/or is based on a current instance of time when the adaptive temporal-based prediction score is generated and/or a duration of time between a given instance of time (e.g., a time of issuance of a recommendation) and a current instance of time when the adaptive temporal-based prediction score is generated. The adaptive temporal-based prediction score may change over time based on temporal feature set progression within an input data set used to generate the score. The adaptive temporal-based prediction score may be based on or take into consideration a content type associated with the recommendation. In various examples in the healthcare domain, the adaptive temporal-based prediction score generated using the predictive machine learning model may represent a likelihood that an initial prescription is not picked up, a likelihood that a refill indicated in a previously-issued prescription is not picked up, and/or a likelihood that a specialist consultation (e.g., with a healthcare provider other than that of the recommending computing entity 110C), follow-up appointment (e.g., with a healthcare provider of the recommending computing entity 110C), diagnostic test and/or procedure (e.g., colonoscopy, mammogram), or course of therapy (e.g., physical therapy, mental health therapy) indicated in a referral is not scheduled and/or not attended by the patient. In one such example, the adaptive temporal-based prediction score may be generated for a prescription and/or refill for a given patient for a current time of year (e.g., business quarter) based on past patient behavior within the same time of year (e.g., quarter) in previous years.

The term “predictive machine learning model” may refer to a data construct that describes parameters, hyperparameters, and/or defined operations of a machine learning model that is configured to generate an adaptive temporal-based prediction score for recommendations indicated in a given input data set. According to various embodiments of the present disclosure, the predictive machine learning model may be used to generate an adaptive temporal-based prediction score for each recommendation of one or more recommendations represented in the input data set. Here, the predictive machine learning model may, for a given recommendation represented in the input data set, perform a predictive inference, such as determining a likelihood (e.g., determining a probability) that the given recommendation will not be performed with respect to one or more actions indicated in the recommendation based on features extracted from the input data set. In one example, the predictive machine learning model may be configured to express the likelihood that a recommendation will not be performed based on a plurality of features extracted from the input data set. Here, the predictive machine learning model may undergo a training process using a training dataset in order to identify features and to determine optimal coefficients representing adjustment or weights to apply with respect to the features in order to produce a target probability reflected in the training dataset based on positive and/or negative correlations between the features and the recommendations. A predictive machine learning model may include a data object created by using machine learning to learn to perform a given function (e.g., a prediction) through training with the training dataset. For example, the training process may formulate a mapping function f from input variables x to discrete output variables y.

The term “input data set” may refer to one or more items of data (e.g., associated with real-time or current recommendations) to which the predictive machine learning model may be applied in order to generate the adaptive temporal-based prediction score (e.g., for pending recommendations). In various embodiments, the input data set may originate from one or more data sources and may include one or more different types of data. The input data set may originate at least in part from a remote computing entity, data available to a recommending computing entity, and/or other sources of data. The input data set may include temporal sequence data. The input data set may include recommendation data and other data pertaining to recommendations indicated in the recommendation data, including supplemental data concerning the individuals, the recommendations, the recommending entities, the suggested course of action of the recommendation, and/or entities related to the individual and/or recommendation, which data may be drawn from one or more data sources and connected or matched with the recommendations indicated in the recommendation data.

The term “training data set” may refer to one or more items of data (e.g., associated with past recommendations) that may be used to train the predictive machine learning model in a training phase. The training data set may be used to identify features in the training data set and to determine optimal coefficients representing adjustment or weights to apply with respect to the identified features (e.g., for past recommendations) in order to produce a target probability reflected in the training data set based on positive and/or negative correlations between the features and the recommendations. Features analogous to the features identified in the training data set may then be extracted from an input data and used by the model to generate the adaptive temporal-based prediction score (e.g., for pending recommendations). The training dataset may (e.g., supervised learning via labeled data) or may not (e.g., unsupervised learning via unlabeled data) include classification labels that characterize data in the training dataset (e.g., indications of compliance or non-compliance results associated with past recommendations). As such, a predictive machine learning model may learn known (e.g., supervised) or unknown (e.g., unsupervised) relationships or patterns from the training dataset. The training data set may include historical transaction data, and/or may be generated based on historical transaction data. The training data set may further comprise data other than the historical transaction data, including data from recommending entities and supplemental data concerning the individuals, the recommendations, the recommending entities, the suggested course of action of the recommendation, and/or entities related to the individual and/or recommendation, which data may be drawn from one or more data sources and connected or matched with the recommendations indicated in historical transaction data.

The term “temporal sequence data” may refer to one or more items of data, with each item associated with a representation of a point in time corresponding to occurrence, origination, and/or issuance of the item.

The term “historical transaction data” may refer one or more items of data (e.g., temporal sequence data) representative of transactions recorded (e.g., over time) with respect to past recommendations. The historical transaction data may be used as the training data set for the predictive machine learning model and/or may be used to generate the training data set. The historical transaction data may originate from a single source or a plurality of sources and may include transaction data originating from the single source or the plurality of sources. The historical transaction data may originate at least in part from a claims database, which may maintain records of claims (e.g., submitted for reimbursement by a paying entity) that pertain to various past recommendations. The claims database may be a claims database maintained by the paying entity indicating transactions registered and recorded by a computing system of the paying entity (e.g., in response to interactions with various entities requesting reimbursement). The historical transaction data may originate at least in part from a remote computing entity and may include transaction data recorded by the remote computing entity at some point in the past with respect to past recommendations.

In one example, the historical transaction data may originally comprise historical transaction data from the claims database, the predictive machine learning model may be originally trained based on the historical transaction data from the claims database, the historical transaction data may be updated based on transaction data from the remote computing entity, and the predictive machine learning model may be retrained based on the updated historical transaction data. The historical transaction data and/or a training data set (e.g., based on the historical transaction data) for the predictive machine learning model may be stored in memory (e.g., in a database) and/or continually updated to reflect new historical data concerning past recommendations (e.g., derived from new transaction data) for continual retraining of the machine learning model.

The predictive machine learning model may be trained and/or configured based at least on the past behavior by individuals with respect to recommendations as indicated in the historical transaction data. For example, the compliance or non-compliance results data for the past recommendations may be used to assign target probabilities of non-compliance to each past recommendation in the training data set, and the assigned target probabilities of non-compliance may be used to train the predictive machine learning model. The predictive machine learning model may be configured and/or trained based on a determined time window for each individual representing an average duration of elapsed time between issuance of recommendations for that individual and following of the recommendations by the individual, as indicated in the historical transaction data. The predictive machine learning model may be configured and/or trained based on a determined probability of non-compliance by each user within and/or outside the respective determined time window for each user as indicated in the historical transaction data. For example, the predictive machine learning model may be configured to determine the probability that a given recommendation will not be performed by the individual within the determined time window for that user. Moreover, the predictive machine learning model may be configured and/or trained based on a determined non-compliance threshold representing a duration of elapsed time since issuance of a recommendation at which the probability of non-compliance with respect to the recommendation should reach a maximum likelihood (e.g., 100%), as indicated by and determined from the historical transaction data.

The term “remote computing entity” may refer to a system for executing and/or recording transactions. In one embodiment, a remote computing entity may be a point-of-sale system that captures transaction as they occur (e.g., in real-time and/or near-real-time) at a point of sale (e.g., retail entity). The remote computing entity may record the transaction in response to interaction between the remote computing entity and an individual. An individual may perform actions (e.g., completing a retail sale) that are registered and recorded as transactions by the remote computing entity, and the remote computing entity may maintain transaction data comprising temporal sequence data representative of the transactions recorded by the remote computing entity. For the purposes of illustration, in one example in the healthcare domain, transaction data from the remote computing entity may originate from pharmacies and comprise data concerning pharmacy transactions including sales associated with prescriptions and/or prescription refills, pick-up events registered for prescriptions and/or prescription refills (e.g., records of patients picking up the prescriptions and/or refills), communications with patients concerning the prescriptions and/or prescription refills (e.g., pick up reminders, indications from patients that they do not intend to pick up the prescriptions and/or refills), and/or reversal or cancellation events concerning the prescriptions and/or prescription refills (e.g., cancellation or reversal of the prescriptions and/or refills that were never picked up).

The term “integration system” may refer to a system (e.g., computing system or subsystem) that is configured to collect, process, format, and/or store data from different sources (e.g., data stored by and/or available to a recommending computing entity, real-time transaction data from a remote computing entity, data from entities related to individuals and/or recommendations). The integration system may be configured to generate new and/or update existing recommendation data based on the data from the different sources. Actions represented by transactions recorded in the transaction data from the remote computing entity may have been performed by an individual in accordance with recommendations issued for the individual. Thus, in various embodiments, the integration system may be configured to update existing recommendation data for pending recommendations by matching and/or reconciling data originating from a recommending computing entity with new transaction data from a remote computing entity based on identifying data pertaining to individuals and/or recommendations within the data from each source. In one example, the integration system may generate new and/or update existing recommendation data based on data stored by and/or available to a recommending computing entity (e.g., by generating new recommendation data pertaining to recommendations newly generated by the recommending computing entity and/or updating existing recommendation data to include newly generated recommendations from the recommending computing entity).

The term “real-time recommendation data” may refer to recommendation data reflecting up-to-date data about recommendations that is available in real-time. The integration system may generate and/or update the real-time recommendation data by incorporating newly added and/or modified data from each of the different sources on which the recommendation data is based into the real-time recommendation data on a periodic basis. For example, the integration system may generate and/or update the real-time recommendation data with new and/or modified data from each different source of data on a daily or hourly basis. In another example, the integration system may generate and/or update the real-time recommendation data with new and/or modified data from each different source in response to detecting changes to the data from the different sources. Thus, in various embodiments, the real-time recommendation data may originate at least in part from a variety of sources, including (e.g., real-time) transaction data from the remote computing entity.

In one example, the real-time recommendation data may include both data for prescriptions that have recently been issued by a recommending computing entity (e.g., computing system of a healthcare provider) and may be modified to reflect an updated pending status for prescriptions that have recently been picked up at the pharmacy. In the foregoing example, the former data may originate from the recommending computing entity (e.g., as newly generated prescriptions reflected in electronic health records but not in the transaction data from the pharmacy), while the latter may originate from the pharmacy (e.g., as pick-up transactions registered with respect to the prescriptions), and the real-time recommendation data reflects the most up to date data from each source.

The term “feature” may refer to a data construct that describes a characteristic or attribute within the input data set and/or the training data set, such as the real-time recommendation data, historical transaction data (e.g., pertaining to past recommendations) used to train the model, and/or supplemental data derived from various sources and pertaining to recommendations indicated in the real-time recommendation data and the historical transaction data. In various embodiments, the extracted features may include time data concerning the recommendation (e.g., when the recommendation was issued), data about entities with which the individual must interact in order to follow the recommendation, location data for the individual and for entities with which the individual must interact in order to follow the recommendation, data identifying and/or classifying the recommendation, implementation details concerning the suggested course of action provided in the recommendation, content type classification of the recommendations and associated priority category classifications for the recommendations, data about costs associated with the recommendations, data about the individuals (e.g., demographic data, age, gender, risk data associated with the individual), average duration for each individual of elapsed time between issuance of recommendations and following the recommendations, and/or duration of elapsed time for a given recommendation since issuance of the recommendation and a current time, and the like.

In one example, example features (e.g., extracted from the input data set) may include prescribed medication name, prescription date, pharmacy filling the prescription, zip codes associated with the individual and the pharmacy filling the prescription, a label name of the medication, dosage, quantity, number of days supplied, and number of refills for a medication, estimated distance for the individual to travel to pick up the prescription, priority category associated with the primary utility or disease category of a prescribed medication, medication pricing data, age, gender, and risk adjustment factors (RAF) for the individual, average prescription pickup time for the individual, elapsed time since issuance of the prescription, pharmacy grouping data, number of medications prescribed on the same day for an individual, cost of a prescribed medication after reimbursement, out-of-pocket cash price of a prescribed medication, month and/or quarter of prescription, administration route of a prescribed medication, past medications prescribed to an individual, historical adherence or compliance rates (e.g., by medication), amount spent by the individual within predetermined periods of time (e.g., 30, 60, 180, 365 days), deductible amounts associated with insurance coverage for the individual, insurance plan type and features, diagnosis data for the individual (e.g., primary diagnoses), hierarchical condition categories (HCCs) or other medical codes linked to diagnoses associated with the individual, numbers and types of chronic conditions of the individual, number of type of acute conditions of the individual, number and types of specialists and providers treating the individual, number of claims associated with the individual, recent procedures involving the individual, spending data concerning the individual, emergency-room (ER) utilization data for the individual, behavioral health issues of the individual, data concerning access of the individual to healthcare services, data concerning financial stress of the individual, data concerning lifestyle, loneliness, and/or caregivers for the individual, and/or prior authorization data for the individual, including number of historical prior authorizations, conditions and procedures with prior authorizations, and/or number of approved and denied prior authorizations.

The term “content type” may refer to a data construct that represents a characteristic (e.g., within the recommendation data) of a recommendation, particularly a classification of the recommendation into a content type or group based on attributes common to all recommendations within each group (e.g., utility classifications or recommendation type classifications). In various embodiments, each recommendation may be assigned a content type.

In one example within the healthcare domain, content types represent particular medications and/or types of medication. The content type may be a feature extracted from the input data set and/or the recommendation data. The predictive machine learning model may be configured and/or trained based on content types associated with recommendations represented in the training data set, and the adaptive temporal-based prediction score for a recommendation may be generated based on a content type associated with the recommendation in the input data set and/or recommendation data.

The term “priority category” may refer to a data construct that describes a characteristic within the recommendation data, particularly a classification of different recommendations into priority groups based on attributes common to all recommendations within each group. In various embodiments, each recommendation may be assigned a content type. In turn, each of the different content types may be assigned a priority group associated with a priority value based on risk attributes associated with the members of the group (e.g., content types known to have the most adverse impact on the individual when ignored, content types that are known to be commonly ignored). Each recommendation may be classified in the priority category that its content type is assigned. In one example, the recommendations may be classified into priority categories associated with varying levels of adverse impact on the individual when the recommendations are ignored. The priority category may be a feature extracted from the input data set and/or the recommendation data. The predictive machine learning model may be configured and/or trained based on features extracted from the input data set and/or recommendation data, including a content type associated with a recommendation.

The term “temporal feature set progression” may refer to adjusting, updating, and/or progressing one or more features that describe temporal characteristics of the input data set and/or recommendation data based on a current time and/or a progression from an initial instance (e.g., values of the predefined features at an initial point in time) to a subsequent instance (e.g., values of the predefined features at a subsequent point in time). In various embodiments, the adaptive temporal-based prediction score generated by the predictive machine learning model is updated by the predictive machine learning model based on the temporal feature set progression during an entirety of the duration of elapsed network time since issuance of a recommendation.

In one example, the predictive machine learning model may be configured and/or trained, and the adaptive temporal-based prediction score may be generated, based on a duration of elapsed time since issuance of a recommendation. Thus, an instance of the adaptive temporal-based prediction score generated at an initial time may differ from an instance of the adaptive temporal-based prediction score generated at a subsequent time by virtue of the temporal feature set progression (e.g., an instance of the duration of elapsed time since issuance of the recommendation at the subsequent time being larger relative to the instance of the duration at the initial time).

In another example, for a selected one of the one or more individuals, an average duration of time between issuance of past recommendations and action by the selected individual with respect to the past recommendations may be determined based on the historical transaction data, and the predictive machine learning model may be configured, trained, and/or used based on the determined average duration of time. In this example, an instance of the adaptive temporal-based prediction score generated at an initial time may differ from an instance of the adaptive temporal-based prediction score generated at a subsequent time by virtue of the temporal feature set progression, as an instance of the average duration of time between issuance and action for the individual at an initial time may differ from an instance of the average at a subsequent time (e.g., due to updates to the historical data and/or recommendation data). Here, the initial instance of the score may differ from the subsequent instance of the score also by virtue of the predictive machine learning model comprising an increase of a determined probability of inaction by an individual for each instance of a duration of elapsed time since issuance of a recommendation, with the probability reaching approximately 100% when the duration satisfies (e.g., meets or exceeds) the determined non-compliance threshold.

### III. COMPUTER PROGRAM PRODUCTS, METHODS, AND COMPUTING ENTITIES

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present disclosure may also be implemented as methods, apparatus, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatus, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. EXAMPLE SYSTEM ARCHITECTURE

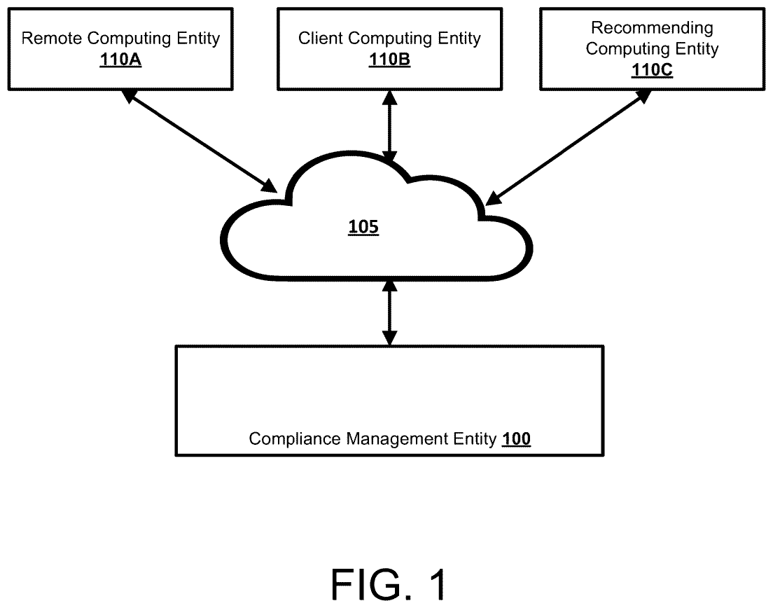

FIG. 1 provides an illustration of an example embodiment of the present disclosure. As shown in FIG. 1, this particular embodiment may include one or more compliance management computing entities 100, one or more remote computing entities 110A, one or more recommending computing entities 110C, and one or more client computing entities 110B. Each of these components, entities, devices, systems, and similar words used herein interchangeably may be in direct or indirect communication with, for example, one another over the same or different wired or wireless networks. Additionally, while FIG. 1 illustrates the various system entities as separate, standalone entities, the various embodiments are not limited to this particular architecture.

In some embodiments, the compliance management computing entity 100 may communicate with one or more client computing entities 110B, one or more remote computing entities 110A, and one or more recommending computing entities 110C using one or more communication networks 105. Examples of communication networks 105 include any wired or wireless communication network including, for example, a wired or wireless local area network (LAN), personal area network (PAN), metropolitan area network (MAN), wide area network (WAN), or the like, as well as any hardware, software and/or firmware required to implement it (.g., network routers, and/or the like).

### A. Example Compliance Management Computing Entity

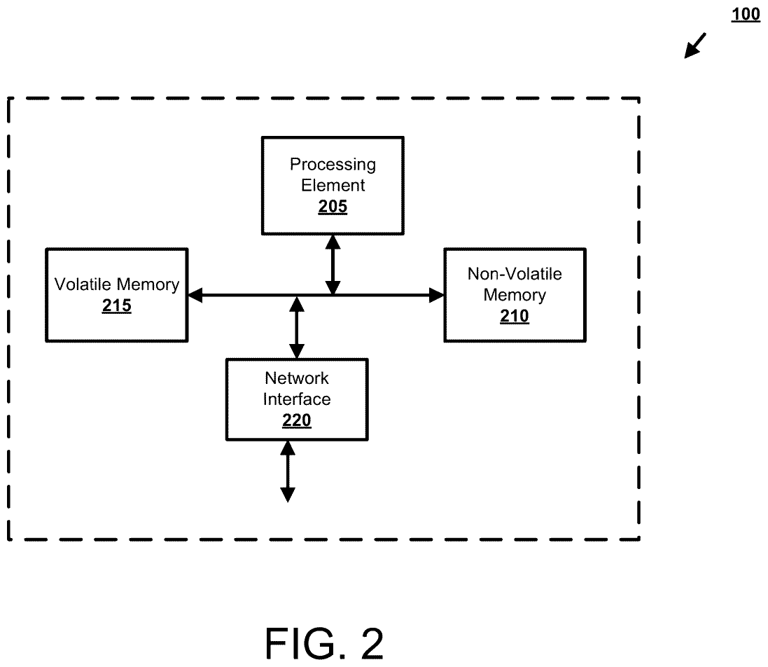

FIG. 2 provides a schematic of an example computing entity such as the compliance management computing entity 100 according embodiments of the present disclosure. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, data, and/or similar terms used herein interchangeably.

As indicated, in one embodiment, the compliance management computing entity 100 may also include one or more communications interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the compliance management computing entity 100 may include, or be in communication with, one or more processing elements 205 (also referred to as processors, processing circuitry, and/or similar terms used herein interchangeably) that communicate with other elements within the compliance management computing entity 100 via a bus, for example. As will be understood, the processing element 205 may be embodied in a number of different ways. For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present disclosure when configured accordingly.

In one embodiment, the compliance management computing entity 100 may further include, or be in communication with, non-volatile media (also referred to as non-volatile storage, memory, memory storage, memory circuitry and/or similar terms used herein interchangeably). In one embodiment, the non-volatile storage or memory may include one or more non-volatile storage or memory media 210, including, but not limited to, hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the compliance management computing entity 100 may further include, or be in communication with, volatile media (also referred to as volatile storage, memory, memory storage, memory circuitry and/or similar terms used herein interchangeably). In one embodiment, the volatile storage or memory may also include one or more volatile storage or memory media 215, including, but not limited to, RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the compliance management computing entity 100 with the assistance of the processing element 205 and operating system.

As indicated, in one embodiment, the compliance management computing entity 100 may also include one or more communications interfaces 220 for communicating with various computing entities, such as by communicating data, content, data, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the compliance management computing entity 100 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the compliance management computing entity 100 may include, or be in communication with, one or more input elements, such as a keyboard input, a mouse input, a touch screen/display input, motion input, movement input, audio input, pointing device input, joystick input, keypad input, and/or the like. The compliance management computing entity 100 may also include, or be in communication with, one or more output elements (not shown), such as audio output, video output, screen/display output, motion output, movement output, and/or the like.

### B. Example Computing Entity

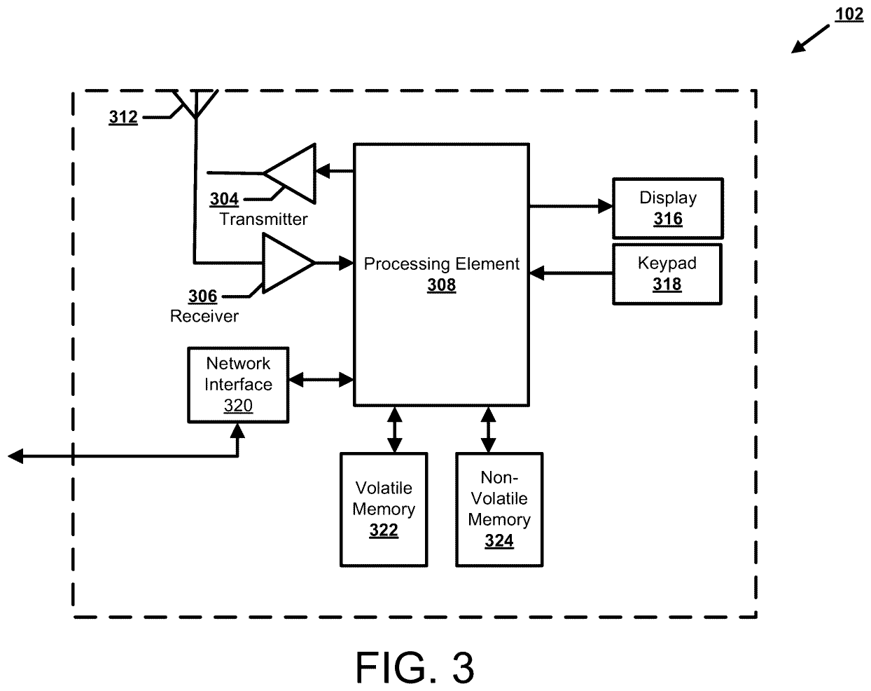

FIG. 3 provides an illustrative schematic representative of a computing entity 102 (e.g., one or more client computing entities 110B, one or more remote computing entities 110A, one or more recommending computing entities 110C, tracking computing entity 101) that can be used in conjunction with embodiments of the present disclosure. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Computing entities 102 can be operated by various parties. As shown in FIG. 3, the computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the computing entity 102 may be capable of operating with one or more air interface standards, communication protocols, modulation types, and access types. More particularly, the computing entity 102 may operate in accordance with any of a number of wireless communication standards and protocols, such as those described above with regard to the compliance management computing entity 100. In a particular embodiment, the computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the computing entity 102 may operate in accordance with multiple wired communication standards and protocols, such as those described above with regard to the compliance management computing entity 100 via a network interface 320.

Via these communication standards and protocols, the computing entity 102 can communicate with various other entities using means such as Unstructured Supplementary Service Data (USSD), Short Message Service (SMS), Multimedia Messaging Service (MMS), Dual-Tone Multi-Frequency Signaling (DTMF), and/or Subscriber Identity Module Dialer (SIM dialer). The computing entity 102 can also download changes, add-ons, and updates, for instance, to its firmware, software (e.g., including executable instructions, applications, program modules), and operating system.

According to one embodiment, the computing entity 102 may include location determining aspects, devices, modules, functionalities, and/or similar words used herein interchangeably. For example, the computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating a position of the computing entity 102 in connection with a variety of other systems, including cellular towers, Wi-Fi access points, and/or the like. Similarly, the computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The computing entity 102 may also comprise a user interface (that can include a display 316 coupled to a processing element 308) and/or a user input interface (coupled to a processing element 308). For example, the user interface may be a user application, browser, user interface, and/or similar words used herein interchangeably executing on and/or accessible via the computing entity 102 to interact with and/or cause display of information/data from the compliance management computing entity 100, as described herein. The user input interface can comprise any of a number of devices or interfaces allowing the computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the computing entity 102 and may include a full set of alphabetic keys or set of keys that may be activated to provide a full set of alphanumeric keys. In addition to providing input, the user input interface can be used, for example, to activate or deactivate certain functions, such as screen savers and/or sleep modes.

The computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the computing entity 102. As indicated, this may include a user application that is resident on the entity or accessible through a browser or other user interface for communicating with the compliance management computing entity 100 and/or various other computing entities.

In another embodiment, the computing entity 102 may include one or more components or functionality that are the same or similar to those of the example compliance management computing entity 100, as described in greater detail above with respect to FIG. 2. As will be recognized, these architectures and descriptions are provided for example purposes only and are not limiting to the various embodiments.

In various embodiments, the computing entity 102 may be embodied as an artificial intelligence (AI) computing entity, such as an Amazon Echo, Amazon Echo Dot, Amazon Show, Google Home, and/or the like. Accordingly, the computing entity 102 may be configured to provide and/or receive information/data from a user via an input/output mechanism, such as a display, a camera, a speaker, a voice-activated input, and/or the like. In certain embodiments, an AI computing entity may include one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. Example System Operations

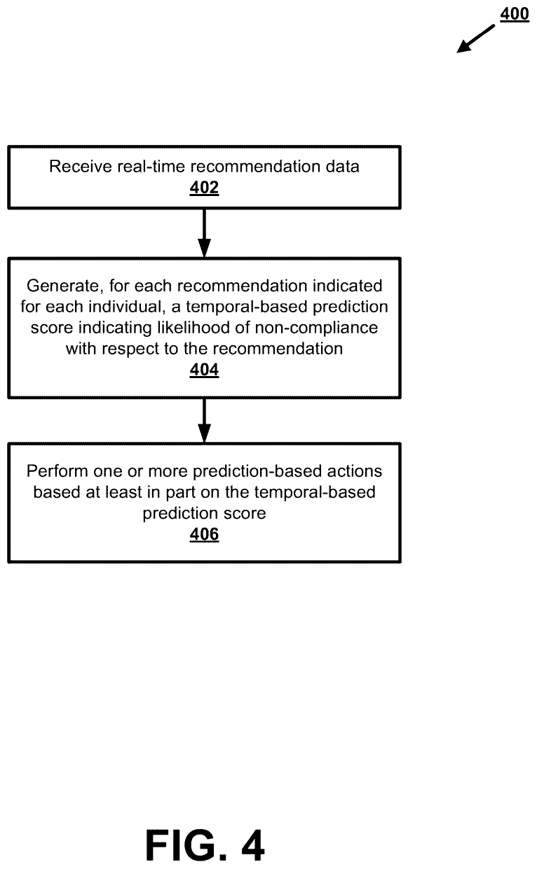

FIG. 4 is a flowchart diagram of an example process 400 for generating adaptive temporal-based prediction scores, according to embodiments herein. Via the various steps/operations of the process 400, the compliance management computing entity 100 can generate adaptive temporal-based prediction scores indicating a likelihood recommendations will not result in compliance and perform prediction-based actions based on the adaptive temporal-based prediction scores.

In some embodiments, a recommendation describes a suggested, proposed, and/or prescribed course of action to be performed by an individual. The recommendation may be issued by the recommending computing entity 110C at a particular point in time (e.g., a recommendation timestamp). A recommendation may include or indicate one or more actions (e.g., to be performed by the individual), including an initial action and follow-up or following actions to be performed subsequent to the initial action. The individual may follow a recommendation by performing an action in accordance with the recommendation (e.g., by performing the suggested course of action of the recommendation). Alternatively, the individual may ignore the recommendation or demonstrate inaction with respect to the recommendation (e.g., by not performing the course of action of the recommendation).

In some embodiments, the recommending computing entity 110C may be a computing system configured to generate recommendations. The recommendations may be generated based on certain data available to the recommending computing entity 110C (e.g., data concerning the individual, data pertinent to a situation of the individual, received user input).

A time of issuance of a recommendation may describe a point in time associated with the recommendation, such as a point of time, such as a point of time at which the recommending computing entity 110C originally generates the recommendation and/or communicates the recommendation to the individual and/or other related entities (e.g., the remote computing entity 110A), and/or a point of time at which a following action of the recommendation is determined to be due and/or communicated to the individual as due.

The process 400 illustrated in FIG. 4 begins at step/operation 402 when the compliance management computing entity 100 receives real-time recommendation data for an individual (e.g., via an integration system 109).

In various embodiments, the integration system 109 may be a system (e.g., computing system or subsystem) that is configured to collect, process, format, and/or store data from different sources (e.g., data stored by and/or available to the recommending computing entity 110C, real-time transaction data from a remote computing entity 110A, data from entities related to individuals and/or recommendations). The integration system 109 may be configured to generate new and/or update existing recommendation data based on the data from the different sources.

Actions represented by transactions recorded in the transaction data from the remote computing entity 110A may have been performed by an individual in accordance with recommendations issued for the individual. Thus, in various embodiments, the integration system 109 may be configured to update existing recommendation data for pending recommendations by matching and/or reconciling data originating from a recommending computing entity 110C with new transaction data from the remote computing entity 110A based on identifying data pertaining to individuals and/or recommendations within the data from each source. In one example, the integration system 109 may generate new and/or update existing recommendation data based on data stored by and/or available to a recommending computing entity 110C (e.g., by generating new recommendation data pertaining to recommendations newly generated by the recommending computing entity 110C and/or updating existing recommendation data to include newly generated recommendations from the recommending computing entity 110C).

The real-time recommendation data may describe recommendation data reflecting up-to-date data about recommendations that is available in real-time. The integration system 109 may generate and/or update the real-time recommendation data by incorporating newly added and/or modified data from each of the different sources on which the recommendation data is based into the real-time recommendation data on a periodic basis. For example, the integration system 109 may generate and/or update the real-time recommendation data with new and/or modified data from each different source of data on a daily or hourly basis. In another example, the integration system 109 may generate and/or update the real-time recommendation data with new and/or modified data from each different source in response to detecting changes to the data from the different sources. Thus, in various embodiments, the real-time recommendation data may originate at least in part from a variety of sources, including (e.g., real-time) transaction data from the remote computing entity 110A.

At step/operation 404, the compliance management computing entity 100 generates, using a predictive machine learning model, for each recommendation of the input data set comprising the recommendation data, for each user profile identifier (e.g., associated with an individual), an adaptive temporal-based prediction score representative of a likelihood of non-compliance with respect to the recommendation (e.g., a likelihood that the one or more actions are not performed).

At step/operation 406, the compliance management computing entity 100 performs one or more prediction-based actions based on the adaptive temporal-based prediction score. In various embodiments, some of the prediction-based actions performed by the compliance management computing entity 100 may be configured to generate and present an compliance interface (and/or other screens/panes) of a graphical user interface of a client computing entity 110B (e.g., rendered on a display of the client computing entity 110B). In various embodiments, the prediction-based actions may include generating and displaying the compliance interface based on the adaptive temporal-based prediction score. The prediction-based actions may also comprise generating outreach priority data based on the adaptive temporal-based prediction score for selected recommendations and presenting the outreach priority data (e.g., via the compliance interface) and/or performing operations based upon the outreach priority data (e.g., determining and/or executing an outreach order based on the priority data with individuals having the highest likelihood of i non-compliance). In another example, the prediction-based actions may include generating and presenting (e.g., via the compliance interface) the adaptive temporal-based prediction score for an individual, in response to detecting generation of a new recommendation by a recommending computing entity 110C.

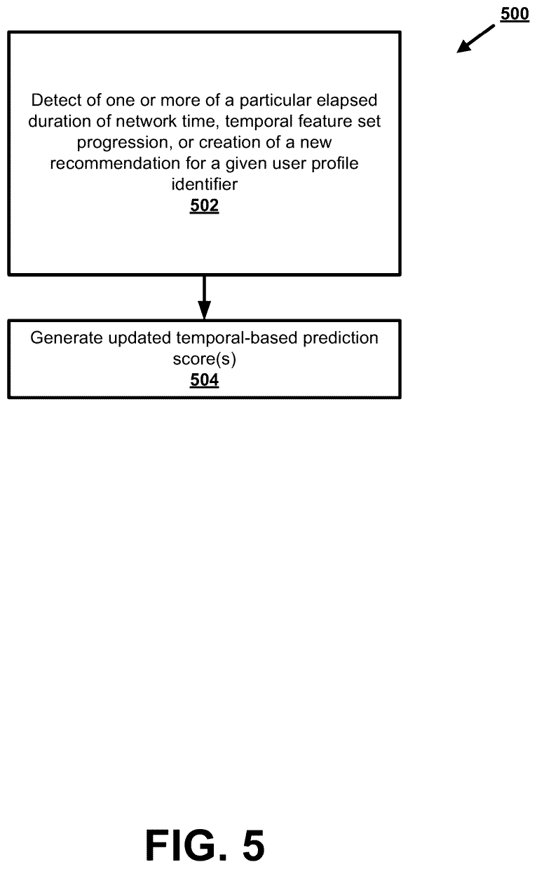

FIG. 5 is a flowchart diagram of an example process 500 for generating adjusted or updated adaptive temporal-based prediction scores. Via the various steps/operations of the process 500, the compliance management computing entity 100 can define, configure, and/or train the predictive machine learning model at least with respect to temporal features of a training data set and a real-time data set and update predictions generated by the predictive machine learning model according to temporal feature set progression.

In various embodiments, a temporal feature set progression may include adjusting, updating, and/or progressing one or more features (e.g., extracted from the input data set) that describe temporal characteristics of the recommendation data based on a current time and/or a progression from an initial instance (e.g., values of the predefined features at an initial point in time) to a subsequent instance (e.g., values of the predefined features at a subsequent point in time).

The process 500 illustrated in FIG. 5 begins at step/operation 502 when the compliance management computing entity 100 detects one or more of a particular elapsed duration of network time (e.g., a day has passed since the last network timestamp associated with an adaptive temporal-based predictive score), temporal feature set progression, or creation of a new recommendation for a given user profile identifier.

At step/operation 504, the compliance management computing entity 100, using the predictive machine learning model, generates the updated adaptive temporal-based prediction score for each recommendation for each user profile identifier.

In various embodiments, the compliance management computing entity 100 may extract the features from the input data set (e.g., including the real-time recommendation data). These extracted features may be analogous to those extracted from the training data set (e.g., including the historical transaction data) and used to train the predictive machine learning model.

The extracted features may include, for each recommendation, the duration of elapsed time since issuance of the recommendation (e.g., as indicated in the real-time recommendation data of the input data set).

Moreover, in various embodiments, the extracted features may include, for each recommendation, a content type classification of the recommendation and an associated priority category classification for the recommendation.

The compliance management computing entity 100 may be configured to perform the update by determining periodically (e.g., daily, hourly) new instances of the adaptive temporal-based prediction scores. Moreover, the compliance management computing entity 100 may be configured to perform the one or more prediction-based actions based on the most recent instance of each of the adaptive temporal-based prediction scores. The adaptive temporal-based prediction score generated in each instance of time may have optimal accuracy with respect to the particular instance of time at which the score is generated.

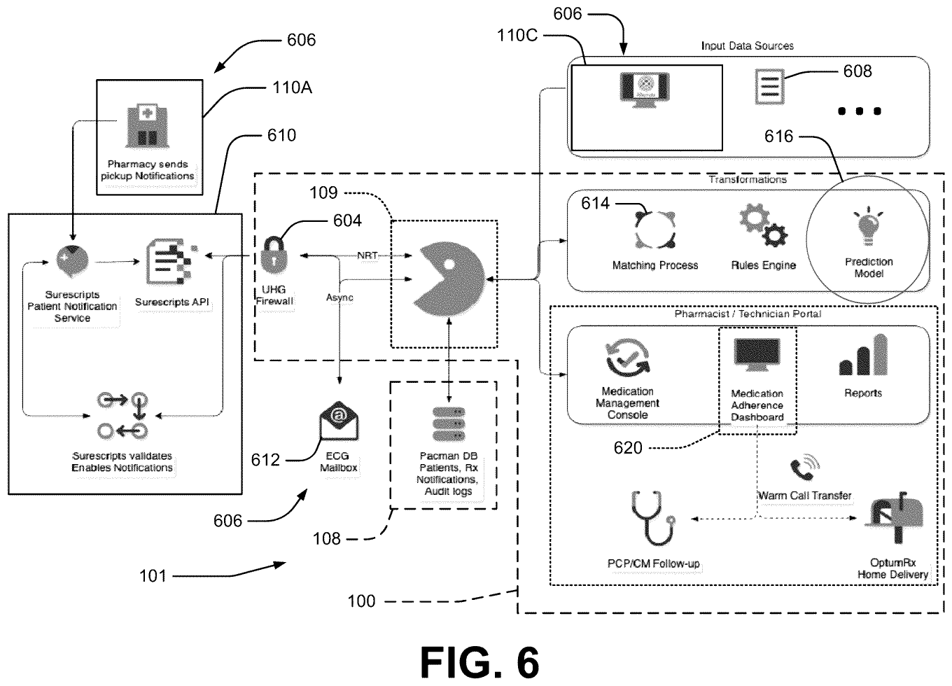

FIG. 6 is a schematic diagram of an example compliance management computing entity 100, according to various embodiments within a healthcare domain.

In the example illustrated in FIG. 6, the compliance management computing entity 100 comprises a compliance tracking entity 106, a storage subsystem 108, and the integration system 109.

According to various embodiments, the integration system 109 may integrate data from various real-time and/or data sources 606. The data sources 606 may include the recommending computing entity 110C (e.g., of the healthcare provider, including data about the individuals, electronic medical records associated with the individuals, newly issued prescription and/or referral data and/or daily prescription reports, any data maintained and/or generated by a practice management system of the healthcare provider) and/or any other additional data sources 606 pertaining to the individual, the prescription or referral, the prescribed medication, general medication data, paying entities (e.g., insurance providers), geographical and/or location data, and/or general medical data (e.g., concerning diagnoses, ailments, levels of risk, treatment best practice), to list a few examples. The data from the recommending computing entity 110C and/or the other data sources 606 may include temporal sequence data representative of one or more recommendations (e.g., prescriptions, referrals, refills) issued by the recommending computing entity 110C, including the date of issuance of each recommendation. In the illustrated example, the data sources 606 comprise the recommending computing entity 110C and a medication data database 608, which provides drug data and drug safety data for various drugs. The data sources 606 may further comprise drug pricing data.

The data sources 606 integrated by the integration system 109 may also include the remote computing entity 110A (e.g., of the pharmacy), and the data collected by the integration system 109 may be (e.g., real-time) transaction data from the remote computing entity 110A. In various embodiments, transaction data from the remote computing entity 110A may originate from the pharmacy and may include a temporal series of data representing data concerning pharmacy transactions including sales associated with prescriptions and/or prescription refills, pick-up events registered for prescriptions and/or prescription refills (e.g., records of patients picking up the prescriptions and/or refills), communications with patients concerning the prescriptions and/or prescription refills (e.g., pick up reminders, indications from patients that they do not intend to pick up the prescriptions and/or refills), and/or reversal or cancellation events concerning the prescriptions and/or prescription refills (e.g., cancellation or reversal of prescriptions and/or refills that were never picked up), along with an indication of an instance of time associated with each transaction (e.g., time and date data indicating when each transaction occurred). The data collected from the remote computing entity 110A (e.g., of the pharmacy) may include drug pricing data (e.g., including cost of drugs to the pharmacy).

In the example illustrated in FIG. 6 the integration system 109 integrates with the remote computing entity 110A (e.g., of the pharmacy) via a firewall system 604 of the compliance management computing entity 100 and an interface system 610.

In various embodiments, the firewall system 604 may be a network security system that monitors and controls incoming and outgoing network traffic according to a predetermined security policy.

Moreover, in various embodiments, the interface system 610 may be a computing entity that provides an interface between the remote computing entity 110A, the recommending computing entity 110C, and/or the compliance management computing entity 100, for sharing data between the different connected entities. For example, the interface system 610 may be a computing entity that collects the (e.g., real-time) transaction data from the remote computing entity 110A (e.g., of the pharmacy). The interface system 610 may perform one or more services with respect to the collected data (e.g., providing a patient notification service, making the transaction data available to other computing systems of other related entities, such as those related to healthcare for patients reflected in the collected historical data). The interface system 610 may provide an application programming interface (API) accessible by the integration system 109 for retrieving the transaction data from the remote computing entity 110A.

The provided API may include a communication protocol for requesting particular types of data from the interface system 610 and/or a validation process for filtering the provided data and/or formatting the provided data according to the needs and/or permissions of the requesting entity (e.g., the integration system 109). In one example, the interface system 610 may provide the transaction data from the remote computing entity 110A appended or formatted to have identifying data for the individuals indicated in the data (e.g., first name, last name, sex and/or gender, age) and filtered such that only the data pertaining to certain individuals is provided to the integration system 109 (e.g., based on matching identifying data first provided by the integration system 109 to the interface system 610 and/or predetermined permissions data associated with the integration system 109).

The data sources 606 integrated by the integration system 109 and/or otherwise accessible by the compliance management computing entity 100 may also include a claims database (not illustrated), which may maintain records of claims (e.g., submitted for reimbursement by a paying entity such as a health insurance provider) that pertain to various past prescriptions (e.g., including refills) and/or referrals. The claims database may be a claims database maintained by the paying entity indicating transactions registered and recorded by a computing system of the paying entity (e.g., in response to interactions with various entities including health care providers and pharmacies requesting reimbursement). The claims data may include (e.g., historical) pharmaceutical claims data and/or medical claims data pertaining to referrals for specialist consultations, services, diagnostic testing and/or procedures, and/or therapy, including information indicative of whether patients scheduled and/or attended healthcare appointments and/or sessions according to recommended courses of action indicated in the referrals. The claims database and/or the claims data may be stored in the storage subsystem 108.

The data sources 606 integrated by the integration system 109 may also include a configuration system 612, as shown in the example illustrated in FIG. 6.

In various embodiments, the configuration system 612 may be a computing system that provides an interface for one or more entities subscribed to a tracking computing entity 101 to provide updated configuration data to a tracking computing entity 101. The configuration system 612 may receive and communicate to the integration system 109 updated member data originating from a subscribed entity concerning which individuals to include and/or exclude in any tracking operations performed for the subscribed entity. In one example, the updated member data may indicate which individuals are no longer customers and/or patients of the subscribed entity and thus should no longer be included in the adherence or compliance tracking operations for the subscribed entity. In another example, the updated member data may indicate which individuals have been newly added as customers and/or patients of the subscribed entity and thus should start being included in the tracking operations for the subscribed entity.

In various embodiments, the integration system 109 may integrate the various data sources 606 on a real-time or near-real-time (NRT) basis and/or asynchronously. For example, the integration system 109 may collect and integrate the data from the remote computing entity 110A (e.g., pharmacy) and/or the recommending computing entity 110C (e.g., healthcare provider) in real-time (e.g., daily, hourly, immediately in response to changes to the data or newly added data), and the integration system 109 may collect and integrate the data from the configuration system 612 asynchronously (e.g., monthly, quarterly).

In various embodiments, the integration system 109 may collect and integrate the data from the various input data sources 606 via a matching process 614 of the compliance management computing entity 100, which is shown in the illustrated example of FIG. 6. The integration system 109 may execute the matching process 614 by matching and/or reconciling data from the different data sources 606 in order to generate combined data representative of the data collected from each of the input data sources 606. The integration system 109 may reconcile the data from each data source 606 such that, in the combined data, data for any individual, recommendation, or other entity indicated in the data from one data source 606 is linked to the data corresponding to the same individual, recommendation, or other entity from the other data source 606, with the most updated data (e.g., being associated with the most recent instance in time) from either data source 606 overwriting any conflicting data from the other data source 606. The integration system 109 may store the combined data in the storage subsystem 108.

In various embodiments, the integration system 109 may generate recommendation data based on data collected from the different input data sources 606. The integration system 109 may store the generated recommendation data in the storage subsystem 108. Here, actions represented by transactions recorded in the transaction data from the remote computing entity 110A (e.g., of the pharmacy) may have been performed by an individual in accordance with recommendations (e.g., prescriptions) issued for the individual by the recommending computing entity 110C (e.g., of the healthcare provider). Thus, in various embodiments, the integration system 109 may collect data from the recommending computing entity 110C (e.g., prescription data from electronic health records (EHRs) of the healthcare provider) and the transaction data from the remote computing entity 110A (e.g., of the pharmacy) and match identifying data pertaining to individuals and identifying data pertaining to recommendations (e.g., prescriptions) in the data from each data source 606 in order to generate combined recommendation data.

In various embodiments, the integration system 109 may generate real-time recommendation data based on data collected and integrated from the remote computing entity 110A (e.g., pharmacy) and the recommending computing entity 110C (e.g., healthcare provider) in real-time (e.g., daily, hourly, immediately in response to changes to the data or newly added data). The integration system 109 may generate real-time recommendation data based on combined data collected from the recommending computing entity 110C (e.g., of the healthcare provider) and the remote computing entity 110A (e.g., of the pharmacy). In one example, the integration system 109 may generate the real-time recommendation data by updating existing recommendation data to include prescriptions newly generated by the recommending computing entity 110C. In another example, the integration system 109 may generate the real-time recommendation data by updating existing recommendation data to reflect newly received transaction data from the pharmacy indicating that certain prescriptions that previously had a status of pending have now been picked up. In one example, the real-time recommendation data may include both data for prescriptions that have recently been issued by the recommending computing entity 110C (e.g., of a healthcare provider) and may also be updated to reflect data for prescriptions that have recently been picked up at the pharmacy (e.g., as indicated in the transaction data from the remote computing entity 110A of the pharmacy). In the foregoing example, the data for prescriptions that have recently been issued may originate from the recommending computing entity 110C (e.g., as newly generated prescriptions reflected in electronic health records), while the updates to the recommendation data (e.g., to remove non-pending prescriptions that have been picked up) due to prescriptions that have recently been picked may originate from the pharmacy (e.g., as pick-up transactions registered with respect to the prescriptions), and the real-time recommendation data may include the most up to date data from each source.

By way of example, a prominent blind spot for healthcare providers is when newly prescribed medications are not picked up from the pharmacy by the patient in a timely manner. Many patients (particularly geriatric patients) are faced with having to manage the pick-up and administration of several medications, sometimes for chronic ailments, and many such medications are prescribed on a recurring basis (e.g., renewals). In addition, providers often face challenges in sustaining sufficiently high medication possession ratios, which often inform important evaluations of the providers, such as the Sustainability, Tracking, Assessment & Rating System (STARS).

Pharmacies have point-of-sale data available that indicates when medications are physically picked up by patients. Reconciling prescription data from the provider's electronic health record (e.g., EHR) with the point-of-sale data from the pharmacies allows providers and/or other adherence or compliance technicians to be informed in near real-time of when patients actually pick up their medications and identify the opportunities to intervene when they do not. In order to provide timely insights to healthcare providers, the electronic prescriptions from the providers' EHR system may be reconciled daily against point-of-sale pharmacy data to identify when a critical medication is not picked up by the patient.

Accordingly, in various embodiments, the tracking computing entity 101 may track whether prescriptions for certain patients and/or for specific classes of medications are picked up by those patients in a timely manner. Here, the tracking computing entity 101 (e.g., via the integration system 109) may receive daily data feeds indicating which prescription orders have been filled and picked up by the patient. In one example, this may include receiving prescription pick-up data from the pharmacy, matching the data to its associated prescription (as previously described), and producing daily reports on prescriptions that have not been picked up by the patient. Moreover, the tracking computing entity 101 may collect data from different data sources 606 in order to approximate a “whole person view” of the individual and their medical situation (e.g., with results in real time), including data from domains such as medical claims and diagnosis codes, hierarchical condition categories (HCCs) or other medical codes linked to diagnoses associated with the individual, risk adjustment factor (RAF) scores for the individual, social determinants of health (SDOH) data (e.g., based on the location of the individual), and physician ratings and/or performance data, to list a few examples.

Moreover, in various embodiments, the tracking computing entity 101 may receive new and/or pending recommendations (e.g., prescriptions) from the recommending computing entity 110C (e.g., an EHR system of a healthcare provider) and score each prescription with a prediction of how likely the prescription is to not be picked up by the patient. The score may be used by the tracking computing entity 101 to prioritize outreach to different patients with respect to different prescriptions, increasing the overall effectiveness and efficiency of the tracking computing entity 101.

A predictive machine learning model 616, such as that included in the example illustrated in FIG. 6, may be used to generate the scores. The predictive machine learning model 616 may estimate risk at a prescription and/or medication level across all content types (e.g., medications) of each prescription for each individual, for example, by generating, for each recommendation for each individual, an adaptive temporal-based prediction score associated with the content type of the recommendation (e.g., a probability score specifically associated with a particular medication or type of medication). Thus, the predictive machine learning model 616 may generate individual estimates for each prescription issued for an individual, accounting for the tendency for there to be, for any individual, certain medications that they are more likely to pick up than others. Moreover, the predictive machine learning model 616 may incorporate and track the impact of progression of time since issuance of the prescription in order to update its predictions over time. The predictive machine learning model 616 may allow live tracking of medications and updating of predictions for tracking, with maximal accuracy, which individuals are likely to miss a medication and continually update the predictions as more time passes since the prescription is written. The tracking computing entity 101 may collect the data from the various data sources 606 in real-time, may provide prediction results in real-time, and may continuously retrain the predictive machine learning model 616 with real-time and/or near-real-time data concerning prescriptions issued for the individual and the individual's behavior with respect to the prescriptions. The predictive machine learning model 616 may be configured such that historical data used to train the model may be comparable with real-time data used to generate the predictions. The compliance management computing entity 100 may be configured to continually feed the real-time data back into retraining the predictive machine learning model 616 as new data is collected.

In various embodiments, the compliance management computing entity 100 uses the predictive machine learning model 616 to generate adaptive temporal-based prediction scores for each recommendation (e.g., prescription, prescription refill, referral) for each individual (e.g., patient) indicated in the real-time recommendation data generated by the integration system 109 and/or stored in the storage subsystem 108. The predictive machine learning model 616 may be configured, trained, and/or used to generate the adaptive temporal-based prediction scores in the manner previously described with respect to the processes illustrated in FIGS. 4 and 5.

The a compliance management computing entity 100 may perform various operations aimed at improving recommendation compliance for an individual and/or a group of individuals, including generating system alerts and reminders, generating and/or sending electronic communications and/or reminders, displaying and/or gathering data relevant to improving compliance (e.g., more detailed data about the recommendations, data concerning the individual's intentions and/or motivations with respect to the recommendations), automated scheduling and calendaring, and/or generating alternative recommendations for the individuals. Any of these operations may be performed based on the adaptive temporal-based prediction scores generated via the predictive machine learning model 616.

More particularly, in various embodiments, the compliance management computing entity 100 may perform one or more prediction-based actions based on the adaptive temporal-based prediction score in the manner previously described with respect to the process illustrated in FIG. 4. Compliance management computing entity 100 is configured to generate and present a compliance interface 620 (and/or other screens/panes), of a graphical user interface of a client computing entity 110B (e.g., rendered on a display of the client computing entity 110B).

In various embodiments, compliance management computing entity 100 may execute communication actions with respect to individuals (e.g., patients). The communication actions may include automatically establishing new communication sessions (e.g., phone calls, messaging sessions) between a patient and a communication system of a healthcare provider (e.g., the healthcare provider whose recommending computing entity 110C generated the prescription in question, a primary care provider) and/or transferring existing communication sessions with the patient to the communication system of the healthcare provider, as indicated in the illustrated example of FIG. 6. The communication actions may also include automatically establishing new communication sessions between a patient and a communication system of an alternative service entity (e.g., a home delivery service for medications, as an alternative to the pharmacy) and/or transferring existing communication sessions with the patient to the communication system of the alternative service entity, as indicated in the illustrated example of FIG. 6.

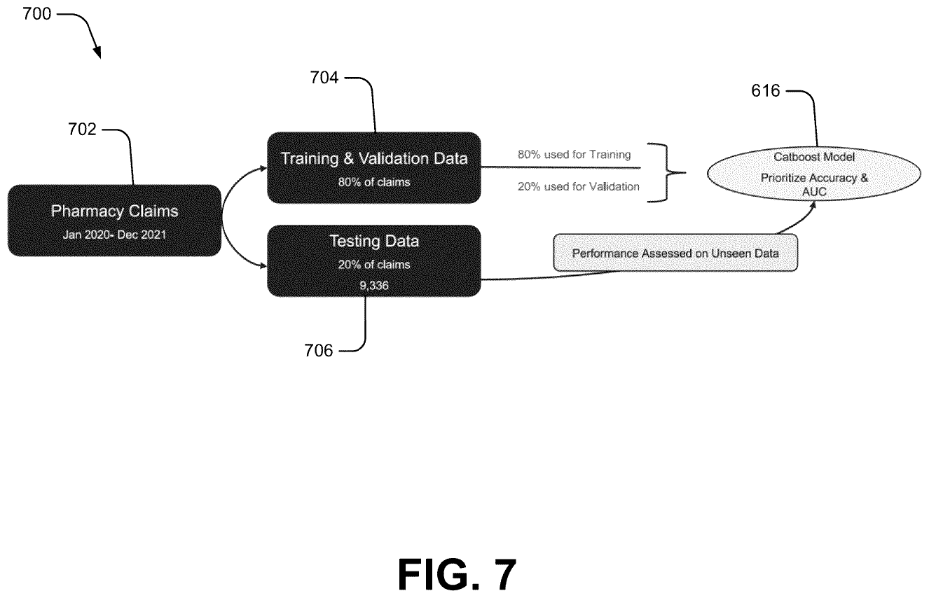

FIG. 7 is an illustration of an example training process, according to embodiments herein. In various embodiments, the predictive machine learning model 616 may be trained based on a training data set, which may include historical transaction data (e.g., pharmaceutical claims 702) and/or other historical data to build base feature sets (e.g., of predefined features) that allow for predictions with respect to the likelihood of compliance to be generated based on real-time recommendation (e.g., prescription) data at the recommendation level (e.g., for each recommendation indicated). The predictive machine learning model 616 may be trained based on the historical transaction data (e.g., pharmaceutical claims data from the claims database), data from recommending computing entities 110C (e.g., EHR records, individual history data, and/or supplemental data concerning the individuals, the recommendations, the recommending entities, the suggested course of action of the recommendation, and/or entities related to the individual and/or recommendation), which data may be drawn from one or more data sources and connected or matched with the recommendations indicated in the historical transaction data, for example.

In the illustrated example, an example training process 700 is based on pharmaceutical claims 702 for a specified date range (e.g., from January of 2020 through December of 2021). In various embodiments, to train the model, the compliance management computing entity 100 may process the pharmaceutical claims 702, including analyzing a claims processing sequence, to determine which of the claims have been paid and which claims have been reversed. For example, for claims that are ultimately paid and picked up, the most recent version of the claim may be flagged as picked up. For claims that are ultimately reversed, the last version of the claim that had a status of paid may be flagged as a reversed claim and used by the predictive machine learning model 616 in the prediction. The features identified within and extracted from such data may be used to configure and/or train the model that generates the scores.

For each claim, information that matches the prescription data that will be given to the predictive machine learning model 616 for scoring, is extracted. Features for the predictive machine learning model 616 include, in some examples, a) the date a prescription is written or generated, b) the pharmacy filling the prescription, c) the zip code of the member and the pharmacy with the prescription, d) the label name of the medication, e) the dosage and quantity of the medication, f) number of days supplied, g) the number of refills remaining, and/or h) travel distance (e.g., the model uses the central longitude and latitudes for the member zip code and pharmacy zip codes to calculate the haversine distance to get an estimated travel distance for each member).

In various embodiments, the predictive machine learning model 616 may be created, as follows:
For each prescription, new fields are calculated or added for the predictive machine learning model 616 in addition to the extracted information discussed above.
The generic product identifier (GPI) is associated with eight priority categories as requested by a compliance management computing entity (e.g., healthcare provider). In one example, these eight categories are predefined and based on primary utility or disease category of the medication. This feature creates categories that have been identified as having the greatest impact on patients should the patients fail to pick up prescribed medication.
Additional information about the cost of medications to the pharmacy is extracted and used from collected drug pricing information.
Patient-specific information, including the age, gender, and the risk adjustment factor (RAF) scores for the patient are compiled and used.
Additionally, features from medical and pharmacy claims history, and patient demographics related to social determinants of health are mapped to the predictive machine learning model 616 to improve the overall accuracy of the model in identifying individual prescriptions with high likelihood of non-compliance.
As time goes on, prescriptions become overall less likely to be picked up, so the overall risk is expected to increase. Another feature in the model is based on the time from issuance of the prescription to pick-up of the prescription. Prescriptions become less likely to be picked up the longer it has been since the prescription has been written. By leveraging how long it has been since the prescription was written and the current date the model is better able to predict the likelihood a patient will pick up the medication based on how long the prescription has been available for pickup by the patient. This will allow for live updating of medication adherence or compliance as more time passes from when a prescription is written if a patient has still not picked up the medication.

In the training process 700 illustrated in FIG. 7, the pharmaceutical claims 702 are divided into training and validation data 704 (80% of the pharmaceutical claims 702) and testing data 706 (20% of the pharmaceutical claims 702). The training and validation data 704 is further divided, with 80% used for training and 20% used for validation. The predictive machine learning model 616 is then fed the training data (e.g., in order to determine parameters of interest) and then validated using the validation data (e.g., in order to optimize configuration of the predictive machine learning model 616 with respect to the parameters). The resulting trained and predictive machine learning model 616 may be configured to produce optimal performance with respect to various metrics, such as accuracy and/or AOC, to name a few examples. The predictive machine learning model 616 is then tested with respect to the testing data 706.

In one example, in the healthcare domain, example features extracted from the training data set and/or input data set may include: prescribed medication name, prescription date, pharmacy filling the prescription, zip codes associated with the individual and the pharmacy filling the prescription, a label name of the medication, dosage, quantity, number of days supplied, and number of refills for a medication, estimated distance for the individual to travel to pick up the prescription, medication category data, priority category associated with the primary utility or disease category of a prescribed medication, medication pricing data, age, gender, and risk adjustment factors (RAF) for the individual, average prescription pickup time for the individual, elapsed time since issuance of the prescription, pharmacy grouping data, number of medications currently prescribed to the individual, number of medications prescribed on the same day for an individual, cost of a prescribed medication after reimbursement, out-of-pocket cash price of a prescribed medication, month and/or quarter of prescription, administration route of a prescribed medication, past medications prescribed to an individual, historical adherence or compliance rates (e.g., by medication), spending data for the individual with respect to predetermined periods of time (e.g., 30, 60, 180, 365 days), deductible amounts associated with insurance coverage for the individual, insurance plan type and features, diagnosis data for the individual (e.g., primary diagnoses), hierarchical condition categories (HCCs) or other medical codes linked to diagnoses associated with the individual, physician ratings and/or performance data, numbers and types of chronic conditions of the individual, number of type of acute conditions of the individual, number and types of specialists and providers treating the individual, number of claims associated with the individual, recent procedures involving the individual, features of spending data concerning the individual, emergency-room (ER) utilization data for the individual, behavioral health issues of the individual, SDOH data pertaining to the individual, data about a region where the individual lives or receives healthcare services, data concerning access of the individual to healthcare services, data concerning financial stress of the individual, data concerning lifestyle, loneliness, and/or caregivers for the individual, and/or prior authorization data for the individual, including number of historical prior authorizations, conditions and procedures with prior authorizations, and/or number of approved and denied prior authorizations. The estimated distance for each individual to travel to pick up the prescription may be generated by determining a haversine distance using central longitude and latitude of an individual's zip code and a zip code of the pharmacy filling the prescription.

In various embodiments, identified and extracted features may include features derived from medical and pharmacy claims history and demographic data concerning the individuals related to SDOH. These features may be used to improve an overall accuracy of the predictive machine learning model in identifying individual prescriptions with a high likelihood of non-compliance.

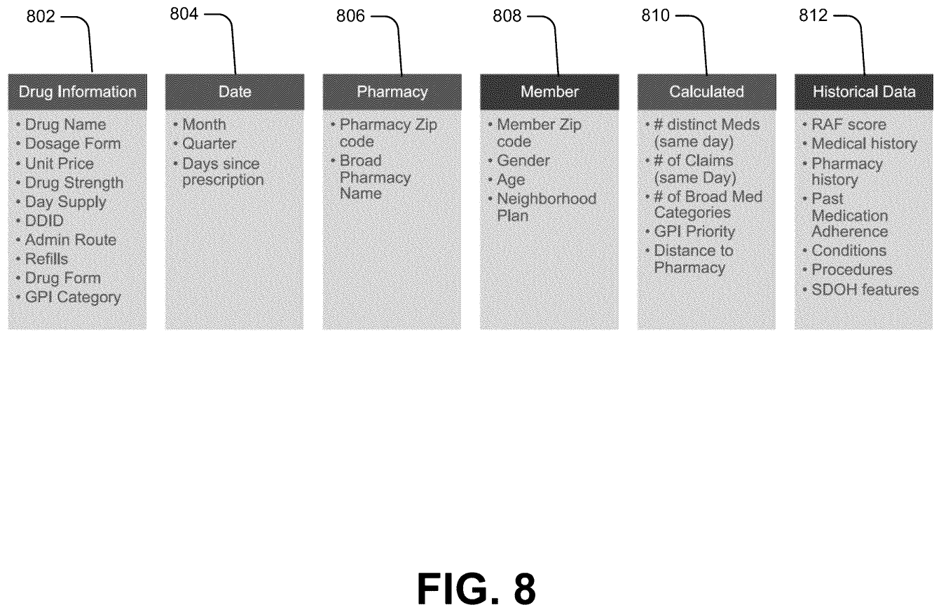

FIG. 8 illustrates example features, according to embodiments herein. In one example, during the training process, the features may be identified in the training data set with respect to past recommendations, and optimal coefficients representing adjustment or weights to apply with respect to the identified features may be determined in order to produce a target probability reflected in the training data set based on positive and/or negative correlations between the features and the recommendations. Features analogous to the features identified in the training data set may then be extracted from the input data and used by the model to generate the adaptive temporal-based prediction score with respect to pending recommendations.

In the illustrated example, the features comprise drug data 802, date data 804, pharmacy data 806, individual data 808, determined data 810, and historical data 812.

Example drug data 802 includes the drug name, dosage form, unit price, drug strength, day supply, dispensable drug identifier (DDID), administration route, refills, drug form, and GPI category (e.g., drug group, drug class, or drug sub-class).

Example date data 804 includes a month of issuance of the prescription, a quarter of issuance of the prescription, and a number of days since issuance of the prescription.

Example pharmacy data 806 includes a pharmacy zip code for the pharmacy filling the prescription and a name of the pharmacy filling the prescription.

Example individual data 808 includes a zip code corresponding to a home of the individual, a gender and/or sex for the individual, an age of the individual, and neighborhood plan data corresponding to the individual.

Example determined data 810 includes a number of distinct medications prescribed for the individual on the same day, a number of claims for prescriptions generated for the individual on the same day, a number of broad medical categories of medications prescribed for the individual, a GPI priority category associated with the GPI category indicated in the drug data 802, and a determined distance from the home of the individual to the pharmacy filling the prescription.

Example historical data 812 includes the RAF score for the individual (e.g., indicating a level of disease burden), medical history data, pharmacy history data, past medication adherence or compliance data, data on conditions affecting the health of the individual, data on procedures performed with respect to the individual, and SDOH data.

Regarding the GPI category of the drug data 802, and the GPI priority of the determined data 810, in various embodiments, one or more of the features of the input data and/or training data used by the compliance management computing entity 100 to generate the adaptive temporal-based prediction score may describe priority categories assigned to the recommendations indicated in the recommendation data. The priority category may represent a classification of different recommendations into content types or groups based on attributes common to all recommendations within each group (e.g., utility classifications or recommendation type classifications). In various embodiments, each recommendation may be assigned a content type. In turn, each of the different content types may be assigned a priority group associated with a priority value based on risk attributes associated with the members of the group (e.g., content types known to have the most adverse impact on the individual when an associated recommendation is not complied with; e.g., content types that are known to have low compliance). Each recommendation may be classified in the priority category that its content type is assigned. The priority category may be a feature extracted from the input data set and/or the recommendation data. The predictive machine learning model may be configured and/or trained based on features extracted from the input data set and/or recommendation data, including a content type associated with a recommendation.

In the healthcare domain context, generally, the content type may be indicative of a utility classification for a medication and/or a disease group. In the context of the illustrated example, in various embodiments, the content type is represented by the GPI category, and each GPI category assigned to each prescription may represent a broad class of drugs (e.g., based on what the drug is used for, what diseases the drug is used to treat, or other characteristics of the drug) and may be determined by which GPI category includes the particular prescribed drug within the GPI classification data. In turn, each GPI priority assigned to each prescription may be a predefined priority score or value representing an importance of adherence or compliance with respect to that GPI category, with different values representing varying levels of importance of adherence or compliance.

In one example, the drug GPI for each medication may be associated with eight predefined priority categories (e.g., designated by a subscribing entity and/or adherence or compliance technicians), with each of the eight predefined priority categories being based on a primary utility or disease category of the medication, and the eight predefined priority categories being identified as having a maximal impact on individuals should the individuals fail to pick up the prescribed medication.

In various embodiments, the compliance management computing entity 100 may analyze a training set, historical data, and/or recommendation data (concerning past recommendations) in order to determine which features have maximal positive or negative impact on the likelihood that a a recommendation will or will not be complied with. The plurality of predefined features may be determined based at least in part upon this determination. Similarly, the predictive machine learning model may be defined, configured, and/or trained based on this determination.

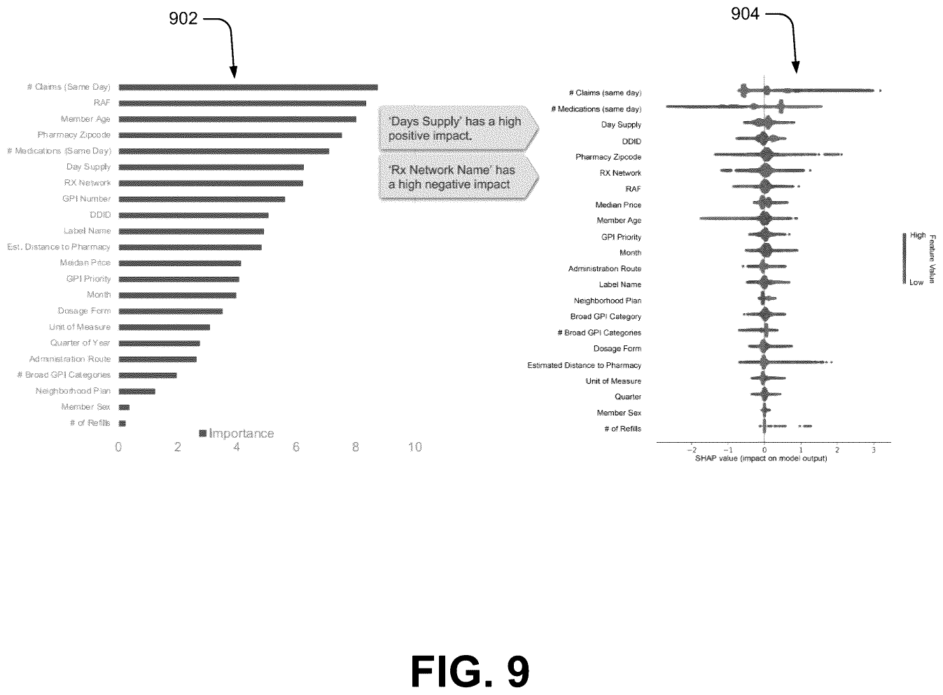

FIG. 9 provides example impact data in accordance with embodiments herein. In FIG. 9, an importance, ranging in value from 1-10, is represented for various features in graph 902. Also in FIG. 9, a degree of positive and negative impact for each of these features is indicated in graph 904.

Graph 902 represents example features in order of importance, from highest to lowest, including: the number of claims for an individual on the same day, the RAF score, the age of the individual, the zip code of the pharmacy, the number of medications prescribed on the same day as the prescription, the days' supply provided in the prescription, the prescription network name, the GPI number, the DDID, the label name of the prescription, the estimated distance between the patient's home and the pharmacy, the median price of the medication, the GPI priority score, the month of the prescription, the dosage form, the unit of measure of the medication, the quarter of the year of the prescription, the administration route, the number of broad GPI categories of medications prescribed to the individual, the neighborhood plan associated with the individual, the sex and/or gender of the individual, and, lastly, the number of refills provided in the prescription.

Graph 904 represents the example features in order of degree of negative or positive impact, from highest to lowest, as: the number of claims on the same day as the prescription, followed by the number of medications prescribed on the same day as the prescription, the days' supply provided in the prescription, the DDID, the zip code of the pharmacy filling the prescription, the prescription network, the RAF score for the individual, the median price of the medication, the age of the individual, the GPI priority score, the month of the prescription, the administration route, the label name of the medication, the neighborhood plan, the GPI category, the number of broad GPI categories prescribed for the individual, the dosage form of the prescription, the estimated distance between the individual's home and the pharmacy filling the prescription, the unit of measure of the prescription, the quarter of the prescription, the sex and/or gender of the individual, and, lastly, the number of refills provided in the prescription. Of these, the days' supply is indicated to have a high positive impact on the likelihood of the individual not complying with a recommendation (e.g., filling and picking up a prescription), while the prescription network name has a high negative impact on the likelihood of the individual not complying, to provide a few examples.

In various embodiments, the plurality of features extracted from the real-time recommendation data and/or training data set may be determined based at least in part upon example impact data provided in graphs 902 and 904. The predictive machine learning model may be defined, configured, and/or trained based on the impact data provided in graphs 902 and 904.

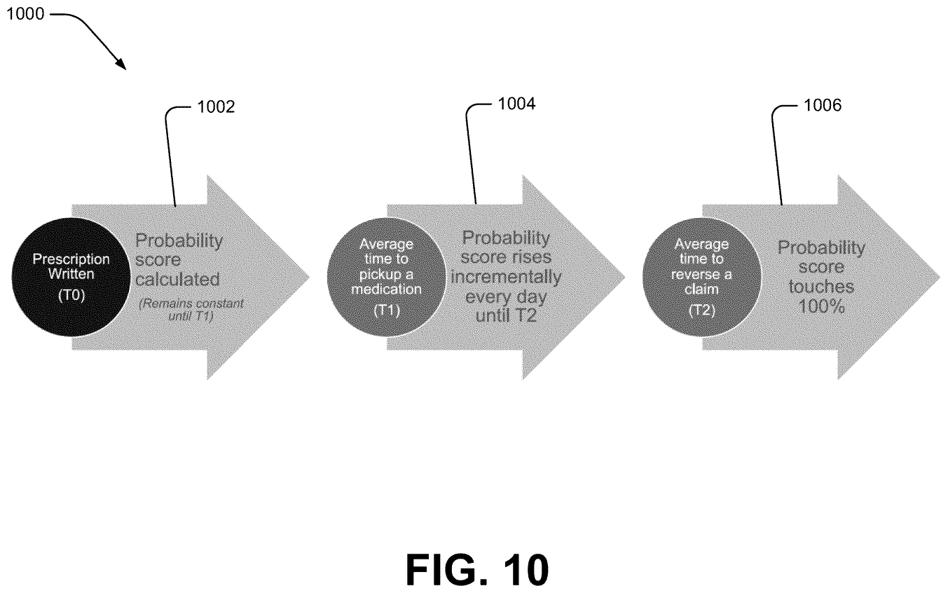

FIG. 10 illustrates example adjustments to adaptive temporal-based prediction scores in accordance with embodiments herein The illustrated process comprises example operations 1002, 1004, 1006, which occur at instances of time represented by T0, T1, and T2, respectively.

The process 1000 begins at step/operation 1002, when a new recommendation is generated (e.g., a prescription is created or written) for an individual (e.g., associated with a user profile identifier) at an instance of time represented by T0 (e.g., a timestamp associated with time T0). The compliance management computing entity 100 generates an adaptive temporal-based prediction score indicating a likelihood of non-compliance associated with the recommendation and the individual, in response to the generation of the new recommendation at T0.

In various embodiments, the compliance management computing entity 100 is configured to determine, for each individual (e.g., associated with a user profile identifier), an average window of time (e.g., number of days) within which the individual historically or usually satisfies compliance with a recommendation, and, in the illustrated example, the adaptive temporal-based prediction score generated in step 1002 reflects the probability of non-compliance of the individual within that average time window. In the illustrated example, the subsequent instance of time T1 corresponds to an end point of the average time window determined for the individual. Accordingly, the adaptive temporal-based prediction score determined at T0 in step 1002 remains constant until T1.

However, once outside the average time window, the likelihood that the individual will comply with the recommendation decreases. Such a tendency might be reflected in the historical transaction data of the training data set and/or the configuration of the predictive machine learning model.

Thus, in step/operation 1004, which occurs at time T1, the duration of elapsed network time since generation of the recommendation reaches a value represented by the difference between T0 and T1 (e.g., T1−T0), which corresponds to the extent of the average time window determined for the individual to comply with the recommendation. In response, the compliance management computing entity 100, at each discrete instance of time between T1 and a subsequent instance of time T2, generates a new instance of the adaptive temporal-based prediction score that is incrementally higher than the previous instance of the score. That is, starting at the original instance of the score determined at time T0 (and any intervening instances of the score determined between T0 and T1, which remain constant), as each subsequent instance of time is associated with a higher duration of time since generation of the recommendation and a further deviance from the average compliance window determined for the individual.

Here, T2 represents a determined non-compliance threshold (e.g., representing the average time it takes for a recommendation to be reversed or canceled). Accordingly, at T2 and all subsequent instances of time, the likelihood that the individual will comply is approximately 0%, as reflected in the historical transaction data of the training data set and/or the configuration of the predictive machine learning model.

Thus, in step/operation 1006, which occurs at T2, the duration of elapsed time since issuance of the prescription reaches a value represented by the difference between T0 and T2 (e.g., T2−T0), which corresponds to the extent of the determined non-compliance threshold. In response, the compliance management computing entity 100, at each discrete instance of time starting at T2 until the recommendation is complied with or reversed/canceled, generates a new instance of the adaptive temporal-based prediction score that reflects a probability of non-compliance that is approximately 100%.

The steps of process 1000 may be performed for each content type (e.g., medication, referral, appointment scheduling, and the like) of each recommendation for each individual. For example, each adaptive temporal-based prediction score may be associated with a content type. Updating the adaptive temporal-based prediction score based on temporal feature set progression during an entirety of the duration of elapsed time since issuance of the recommendation (e.g., as described with respect to steps 1004 and 1006) may include variations based on the content type at given segments of the duration of elapsed time.

In various embodiments, this maximum adaptive temporal-based prediction score may be used to identify individuals most in need of outreach and to schedule and/or execute communication actions. The compliance management computing entity 100 may be configured to generate and send alerts or notifications in response to the score reaching a maximum value. The alerts or notifications may be automatically sent by the compliance management computing entity 100 to the recommending computing entity 110C and/or the client computing entity 110B, which may present the alerts or notifications to the recommender and/or individual, respectively, providing a well-targeted reminder to the individual and a timely notification to the recommender concerning the individual's failure to comply with the recommendation.

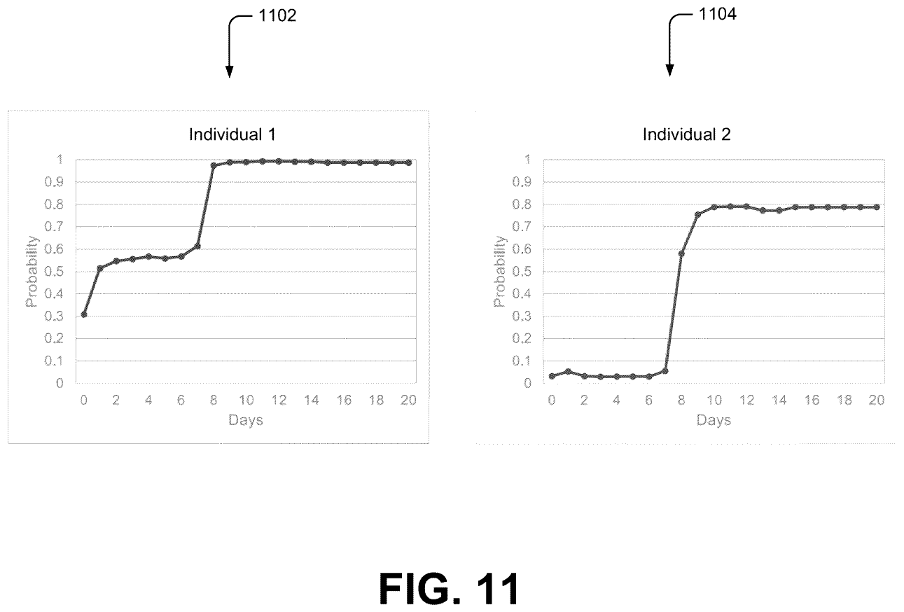

FIG. 11 depicts a comparison of example adaptive temporal-based prediction scores for two different individuals in accordance with embodiments herein. In FIG. 11, graphs 1102, 1104 illustrate how example adaptive temporal-based prediction scores for two different individuals (Individual 1, Individual 2, respectively) can differ with respect to each other and with respect to different instances of time. Each graph 1102, 1104 plots a number of days on the x-axis, representing a duration of time elapsed between the time of generating the adaptive temporal-based prediction score (e.g., based on a timestamp associated therewith) and the provision of a recommendation (e.g., issuance of a prescription; e.g., based on a different timestamp associated therewith). The y-axis of each graph 1102, 1104 represents the adaptive temporal-based prediction score corresponding to each instance of time (e.g., day) represented on the x-axis.

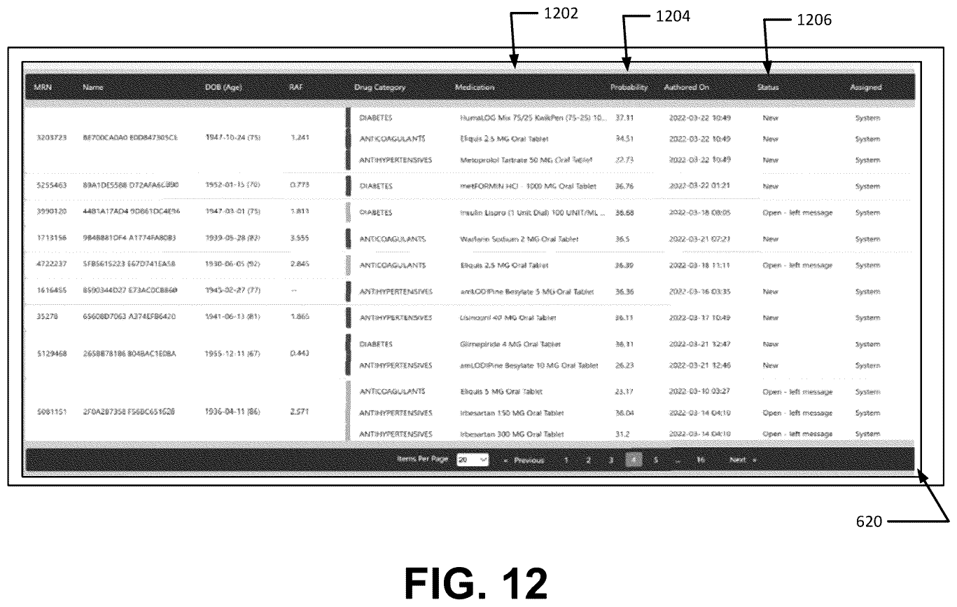

FIG. 12 depicts an example compliance interface 620 in accordance with embodiments herein. In embodiments, a compliance management computing entity 100 may generate the compliance interface 620 and cause the interface 620 to be rendered via a display of a computing entity (e.g., 10A, 110B, 110C). The compliance interface 620 is configured to render visual representations of recommendation data and related data based on adaptive temporal-based prediction scores generated by predictive machine learning model 616. For example, the compliance interface 620 is configured to display the adaptive temporal-based prediction scores as numerical values associated with different individuals (e.g., associated with different user profile identifiers). In another example, the compliance interface 620 is configured to render visual representations of recommendation data based on adherence or compliance outreach priority data generated using the adaptive temporal-based prediction scores.

In the illustrated example, the compliance interface 620 is configured to render recommendation data in table or multi-dimensional matrix format, including a probability column 1204, a recommendation column 1202, and a status column 1206, among others.

In embodiments, the recommendation column 1202 is configured to render identifying data for recommendations indicated in the recommendation data. In the illustrated example, each of the recommendations are represented by the names of prescribed medications.

In embodiments, the probability column 1204 is configured to render the adaptive temporal-based prediction score for each of the recommendations indicated in the recommendation data. In the illustrated example, each of the scores is a numerical value. For example, an adaptive temporal-based prediction score may be determined and displayed for each recommendation for each individual, at the recommendation level. The adaptive temporal-based prediction scores presented in the probability column 1204 also represent the most recent instance of the score (e.g., generated on the same day or during the same network time period as the scores are displayed) and thus represent the most accurate possible score for the point in time at which the score is displayed.

In embodiments, the status column 1206 is configured to render status data for each of the recommendations indicated in the recommendation data. In the illustrated example, some recommendations have a status of “new” indicating that the recommendation is newly generated by the recommending computing entity 110C (e.g., and possibly has not been communicated to related entities such as the remote computing entity 110A). Other recommendations have a status of “open—left message” indicating that the recommendation is still pending (e.g., full compliance has not been performed) and that at least one communication action has been scheduled and/or executed by the compliance management computing entity 100 with respect to the recommendation.

In various embodiments, the recommendations indicated in the compliance interface 620 may be filtered based whether the temporal-based probability score meets or exceeds a priority threshold. In another example, the recommendations indicated in the compliance interface 620 may be displayed in an order based on the temporal-based probability scores, with the recommendations having the highest likelihood of non-compliance displayed more prominently (e.g., higher in list, on the first page). Here, the order may also determine the order in which communication actions (e.g., sending messages to client computing entities 110B concerning the recommendations) are scheduled for transmission or execution by the compliance management computing entity 100.

Accordingly, in various embodiments of the present disclosure, a predictive machine learning model may be defined and trained at least with respect to temporal features of a training data set, and predictions generated by the predictive machine learning models may be updated according to temporal feature set progression. A predictive machine learning model may generate an adaptive temporal-based prediction score that indicates a likelihood of non-compliance with respect to a given recommendation. The predictive machine learning model may be trained based on one or more features (e.g., including temporal features) associated with historical transaction data that are also associated with the recommendations, and/or past compliance associated with past recommendations associated with a user profile identifier. The adaptive temporal-based prediction score is updated, by the predictive machine learning model, based on temporal feature set progression.

### VI. Conclusion

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method comprising:
receiving, by a computing entity, real-time recommendation data associated with a user profile identifier, wherein the real-time recommendation data (i) originates at least in part from a remote computing entity and (ii) comprises temporal sequence data that indicates (a) a recommendation for the user profile identifier, and (b) the recommendation associated with a content type;
generating, by the computing entity and using a predictive machine learning model and based at least in part on (i) a duration of elapsed network time since provision of the recommendation and (ii) the content type associated with the recommendation, an adaptive temporal-based prediction score representative of a likelihood of non-compliance with respect to the recommendation, wherein the predictive machine learning model has been trained based at least in part on: (i) one or more features (a) associated with historical transaction data and (b) associated with the recommendation, or (ii) actions performed in accordance with the historical transaction data associated with the user profile identifier; and
initiating, by the computing entity, the performance of one or more prediction-based actions based at least in part on the adaptive temporal-based prediction score.

2. The computer-implemented method of claim 1, wherein the actions performed in accordance with the historical transaction data associated with the user profile identifier comprise one or more of: compliance with past recommendations, non-compliance with past recommendations, incomplete compliance with past recommendations, or a historical compliance duration based on elapsed time between provision of past recommendations and compliance with the past recommendations.

3. The computer-implemented method of claim 1, wherein the likelihood of non-compliance increases based at least in part on an increase in the duration of elapsed network time.

4. The computer-implemented method of claim 1, wherein the one or more prediction-based actions comprise one or more of causing rendering of an adaptive temporal-based prediction score interface element via a display device, transmission of a non-compliance risk communication, or transmission of a renewed recommendation communication.

5. The computer-implemented method of claim 1, wherein the one or more prediction-based actions are selected based at least in part on the adaptive temporal-based prediction score satisfying a particular threshold.

6. The computer-implemented method of claim 1, further comprising:
updating the adaptive temporal-based prediction score, by the computing entity, using the predictive machine learning model and based at least in part on temporal feature set progression.

7. The computer-implemented method of claim 6, wherein the temporal feature set progression comprises a temporal representation of changes in the one or more features associated with the recommendation during the duration of elapsed network time.

8. An apparatus comprising at least one processor and at least one memory storing instructions that, with the at least one processor, cause the apparatus to:
receive real-time recommendation data associated with a user profile identifier, wherein the real-time recommendation data (i) originates at least in part from a remote computing entity and (ii) comprises temporal sequence data that indicates (a) a recommendation for the user profile identifier, and (b) the recommendation associated with a content type;
generate, using a predictive machine learning model and based at least in part on (i) a duration of elapsed network time since provision of the recommendation and (ii) the content type associated with the recommendation, an adaptive temporal-based prediction score representative of a likelihood of non-compliance with respect to the recommendation, wherein the predictive machine learning model has been trained based at least in part on: (i) one or more features (a) associated with historical transaction data and (b) associated with the recommendation, or (ii) actions performed in accordance with the historical transaction data associated with the user profile identifier; and
initiate the performance of one or more prediction-based actions based at least in part on the adaptive temporal-based prediction score.

9. The apparatus of claim 8, wherein the actions performed in accordance with the historical transaction data associated with the user profile identifier comprise one or more of: compliance with past recommendations, non-compliance with past recommendations, incomplete compliance with past recommendations, or a historical compliance duration based on elapsed time between provision of past recommendations and compliance with the past recommendations.

10. The apparatus of claim 8, wherein the likelihood of non-compliance increases based at least in part on an increase in the duration of elapsed network time.

11. The apparatus of claim 8, wherein the one or more prediction-based actions comprise one or more of causing rendering of an adaptive temporal-based prediction score interface element via a display device, transmission of a non-compliance risk communication, or transmission of a renewed recommendation communication.

12. The apparatus of claim 8, wherein the one or more prediction-based actions are selected based at least in part on the adaptive temporal-based prediction score satisfying a particular threshold.

13. The apparatus of claim 8, wherein the instructions, with the at least one processor, further cause the apparatus to:
update the adaptive temporal-based prediction score, using the predictive machine learning model and based at least in part on temporal feature set progression.

14. The apparatus of claim 13, wherein the temporal feature set progression comprises a temporal representation of changes in the one or more features associated with the recommendation during the duration of elapsed network time.

15. A non-transitory computer-readable storage medium storing instructions that, when executed by at least one processor of an apparatus, cause the apparatus to:
receive real-time recommendation data associated with a user profile identifier, wherein the real-time recommendation data (i) originates at least in part from a remote computing entity and (ii) comprises temporal sequence data that indicates (a) a recommendation for the user profile identifier, and (b) the recommendation associated with a content type;
generate, using a predictive machine learning model and based at least in part on (i) a duration of elapsed network time since provision of the recommendation and (ii) the content type associated with the recommendation, an adaptive temporal-based prediction score representative of a likelihood of non-compliance with respect to the recommendation, wherein the predictive machine learning model has been trained based at least in part on: (i) one or more features (a) associated with historical transaction data and (b) associated with the recommendation, or (ii) actions performed in accordance with the historical transaction data associated with the user profile identifier; and
initiate the performance of one or more prediction-based actions based at least in part on the adaptive temporal-based prediction score.

16. The computer-readable storage medium of claim 15, wherein the actions performed in accordance with the historical transaction data associated with the user profile identifier comprise one or more of: compliance with past recommendations, non-compliance with past recommendations, incomplete compliance with past recommendations, or a historical compliance duration based on elapsed time between provision of past recommendations and compliance with the past recommendations.

17. The computer-readable storage medium of claim 15, wherein the likelihood of non-compliance increases based at least in part on an increase in the duration of elapsed network time.

18. The computer-readable storage medium of claim 15, wherein the one or more prediction-based actions comprise one or more of causing rendering of an adaptive temporal-based prediction score interface element via a display device, transmission of a non-compliance risk communication, or transmission of a renewed recommendation communication.

19. The computer-readable storage medium of claim 15, wherein the one or more prediction-based actions are selected based at least in part on the adaptive temporal-based prediction score satisfying a particular threshold.

20. The computer-readable storage medium of claim 15, wherein the instructions, with the at least one processor, further cause the apparatus to:
update the adaptive temporal-based prediction score, using the predictive machine learning model and based at least in part on temporal feature set progression.
